(19) United States
(12) Patent Application Publication
(10) Pub. No.: US 2026/0022103 A1
(43) Pub. Date: Jan. 22, 2026

(54) PRAMIPEXOLE XINAFOATE AND SUSTAINED-RELEASE PHARMACEUTICAL FORMULATION THEREOF

(71) Applicant: JEWIM PHARMACEUTICAL (SHANDONG) CO., LTD., Taian, Shandong (CN)

(72) Inventors: Yingji SUN, Taian, Shandong (CN); Yuguang WANG, Taian, Shandong (CN); Xiaoyan XING, Taian, Shandong (CN); Shankui LIU, Taian, Shandong (CN); Tiejun LI, Taian, Shandong (CN)

(21) Appl. No.: 18/997,064

(22) PCT Filed: Mar. 14, 2023
(86) PCT No.: PCT/CN2023/081211

§ 371 Date: Jan. 18, 2025

(30) Foreign Application Priority Data
Jul. 26, 2022 (CN) 202210887647.5

(51) Int. Cl. C07D 277/82; A61K 9/00; A61K 9/10; A61K 31/047; A61K 31/167; A61K 31/245; A61K 31/428; A61K 47/02; A61K 47/12; A61K 47/18; A61K 47/26; A61K 47/32; C07C 65/11

(52) U.S. Cl. CPC C07D 277/82; A61K 9/0019; A61K 9/10; A61K 31/047; A61K 31/167; A61K 31/245; A61K 31/428; A61K 47/02; A61K 47/12; A61K 47/183; A61K 47/26; A61K 47/32; C07C 65/11

(57) ABSTRACT

A pramipexole xinafoate and a sustained-release pharmaceutical formulation thereof are provided. The pramipexole is prepared into its xinafoate, enabling reducing a solubility of the pramipexole salts. A suspension liquid formulation prepared from the pramipexole xinafoate has a good sustained-release effect and good stability, and is suitable for large-scale production.

### TECHNICAL FIELD

The present invention relates to the field of pharmaceutical chemical industry, and in particular, a pramipexole xinafoate and a sustained-release pharmaceutical formulation thereof.

### BACKGROUND

Parkinson's disease (PD) is a motor system disorder of the nervous system. It is characterized by a progressive disease that affects movement and results in loss of dopamine-producing brain cells, causing tremors of the hands, arms, legs, jaw and face and/or rigidity or stiffness of the limbs and trunk. The main symptoms include muscle stiffness, motor retardation, resting tremor and postural instability.

Pramipexole has the molecular formula of C₁₁H₁₇N₃S and the chemical name of (S)-(−)-2-amino-6-(propylamino)-4,5,6,7-tetrahydrobenzothiazole. The affinity of the S(−) isomer to a dopamine D2 receptor is about 8-10 times that of the R(+) isomer and 2 times that of a racemate. The therapeutic efficacy of the S(−) isomer of the pramipexole on the PD is also obviously better than that of the R(+) isomer and the raceme. The chemical structural formula of the S(−) isomer of the pramipexole is as follows:

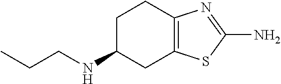

The pramipexole is currently available in the form of oral dosages on the market, such as common immediate-release tablets and sustained-release tablets. However, the pramipexole has a short half-life. Patients with PD need long-term or even lifelong administration. At the same time, the patients with PD are accompanied by the symptoms of tremor, muscular rigidity, bradykinesia and the like. Therefore, oral administration brings serious inconvenience to the patients with PD and their nursing staff.

### SUMMARY

In studies of the pramipexole salts by the present inventors, it is unexpectedly discovered that, the pramipexole salt formed with xinafoic acid has significantly less solubility than that of other acid salts. Upon preparation into a suspension injection, it has a good sustained-release effect through subcutaneous and intramuscular injection, and a stable and effective blood drug level can be maintained for a prolonged period of time.

Embodiments of the present invention are as follows:

A pramipexole xinafoate, having the following structural formula:

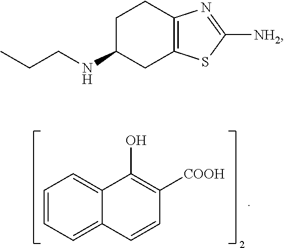

The pramipexole xinafoate of the present invention may be prepared by using the following method:

A pramipexole is dissolved in a reaction solvent, xinafoic acid is added at room temperature to 60° C. for reaction (for 2-4 h), and after the reaction is finished, the reaction product is cooled to room temperature and filtered and separated to obtain a crystal.

The reaction solvent is selected from one or more of alcohols, ketones, nitriles, ethers, dichloromethane and dimethyl sulfoxide.

Preferably, the reaction solvent is selected from anhydrous methanol, anhydrous ethanol, 95% ethanol, isopropanol, benzyl alcohol, acetone or acetonitrile, more preferably anhydrous ethanol.

In the preparation method, the molar ratio of the pramipexole to the xinafoic acid is 1:2.

Further, provided is a sustained-release pharmaceutical formulation of pramipexole, having the pramipexole xinafoate of the present invention as a main pharmaceutical active ingredient.

The sustained-release pharmaceutical formulation may be a capsule, a tablet, a dispersible tablet, an oral suspension, and a suspension injection.

In order to improve the compliance of the pharmaceutical formulation and reduce discomfort brought by injection of the suspension liquid formulation, the pharmaceutical formulation further includes an analgesic, such as one or more of benzyl alcohol, chlorobutanol, procaine hydrochloride and lidocaine.

The suspension liquid formulation further includes one or more of a wetting agent, a suspending agent, an antioxidant, a pH regulator and an osmotic pressure regulator.

Preferably, the wetting agent is selected from one or more of polysorbate, polyoxyethylene castor oil, poloxamer 188 and lecithin; the suspending agent is selected from one or more of PVP, gelatin, methylcellulose, sodium carboxymethylcellulose and polyethylene glycol; the stabilizing agent is selected from one or more of sodium sulfite, sodium bisulfite, sodium metabisulfite, sodium thiosulfate and an EDTA disodium salt; the pH regulator is selected from one or more of hydrochloric acid, sodium hydroxide, sodium bicarbonate, an acetic acid-sodium acetate buffer and a citric acid-sodium citrate buffer; and the osmotic pressure regulator is selected from sodium chloride and/or glucose.

In the sustained-release pharmaceutical formulation of the present invention, the particle size of the pramipexole xinafoate is D₁₀: 0.1-1 μm; D₅₀: 0.5-5 μm; and D₉₀: 2-30 μm. Preferably, the particle size of the crystal is D₁₀: 0.1-0.5 μm; D₅₀: 0.5-1 μm; and D₉₀: 2-10 μm.

A method for preparing the suspension liquid formulation of the present invention is as follows:
1) micronizing the pramipexole xinafoate to a certain particle size distribution;
2) adding the micronized pramipexole xinafoate and a wetting agent into an appropriate amount of water for injection to be fully wetted, prior to addition into a concentrated mixing tank, and sterilizing at 121° C. for 15 min;
3) adding other auxiliary materials into an auxiliary tank, adding an appropriate amount of the water for injection for dissolving, prior to sterilizing by filtration;
4) adding the drug liquor in a concentrated mixing tank and the auxiliary liquor in the auxiliary tank into a diluted mixing tank, and adding the water for injection to a defined volume to obtain a suspension drug liquor; and
5) filling the suspension drug liquor into an ampoule for sealing.

The pramipexole xinafoate of the present invention may be used in preparing a drug for treating Parkinson's disease.

The present invention has the following advantages:

The pramipexole xinafoate of the present invention is prepared into an injection for administration, may achieve long-term stable blood drug level, reduces the administration times, improves the patient compliance, avoids the adverse reaction and the reduction of the drug effect caused by the fluctuation of the blood drug level, and is favorable for better exerting the drug effect.

### BRIEF DESCRIPTION OF THE DRAWINGS

FIG. 1 shows blood drug level curves of injections prepared from different pramipexole salts; and

FIG. 2 shows blood drug level curves of pramipexole xinafoate suspension injections in different formulas.

### DETAILED DESCRIPTION

In the following, the present invention is described in detail with reference to specific embodiments and exemplary examples, but the description should not be construed as limiting the present invention in any way. Those skilled in the art will appreciate that various equivalent substitutions, modifications or improvements may be made to the technical solutions and embodiments of the present invention without departing from the spirit and scope of the present invention, and are within the scope of the present invention. The scope claimed in the present invention is subject to the appended claims. The specific examples are described as follows:

### EXAMPLE 1 PREPARATION OF PRAMIPEXOLE SALTS

1. Preparation of pramipexole xinafoate

Pramipexole (12.6 mmol, 2.6635 g) and 50 mL of anhydrous ethanol were added into a reaction flask and stirred for dissolving, the solution was heated to 50° C., xinafoic acid (25.2 mmol, 4.7446 g) was added and dissolved, prior to continuous stirring for 2 h, after the reaction is finished, the reaction product was cooled to room temperature and subjected to suction filtration, and the filter cake was washed with the anhydrous ethanol for three times (2 mL×3) and dried to dryness at 40-50° C. under reduced pressure to obtain 6.5708 g of pramipexole xinafoate with the yield of 88.74%.
2. Pramipexole hydrochloride, pramipexole benzoate and pramipexole pamoate were prepared respectively with reference to the above method.

### EXAMPLE 2 STUDY OF SOLUBILITY OF PRAMIPEXOLE SALTS

The concentrations of the pramipexole salts in different media for saturation at 48 h were determined by a shake flask dissolution method at 37° C. The results are shown in Table 1.

TABLE 1

| | Saturation | Saturation | Saturation | Saturation |
|---|---|---|---|---|
| | concentra- | concentra- | concentra- | concentra- |
| | tion of | tion of | tion of | tion of |
| | pramipexole | pramipexole | pramipexole | pramipexole |
| | xinafoate | hydrochloride | benzoate | pamoate |
| Media | (μg/ml) | (μg/ml) | (μg/ml) | (μg/ml) |
| Phosphate | 2.64 | 1249.45 | 392.11 | 275.34 |
| buffer at | | | | |
| pH 6.8 | | | | |
| Phosphate | 4.66 | 1363.53 | 427.37 | 345.29 |
| buffer at | | | | |
| pH 7.4 | | | | |
| Water for | 0.78 | 1187.36 | 292.72 | 189.25 |
| injection | | | | |
| 0.9% NaCl | 1.05 | 1207.41 | 325.91 | 227.63 |

The results show the solubility of the pramipexole xinafoate is significantly decreased compared with those of the other salts.

### EXAMPLE 3 PREPARATION OF PRAMIPEXOLE XINAFOATE SUSPENSION INJECTION

For the pramipexole xinafoate, a jet mill was used with the feeding speed set to be 400 rpm and the feeding pressure to be 2.1-2.3 bar, and the crushing pressure was adjusted. The pramipexole xinafoate was crushed to D₁₀: 0.2 μm; D₅₀: 0.6 μm; and D₉₀: 2.1 μm. Referring to Table 2, the micronized pramipexole xinafoate and a wetting agent were added into an appropriate amount of water for injection to be fully wetted, prior to addition into a concentrated mixing tank and sterilizing at 121° C. for 15 min. Other auxiliary materials were added into an auxiliary tank, an appropriate amount of the water for injection was added for dissolving, prior to sterilizing by filtration. The drug liquor in the concentrated mixing tank and the auxiliary liquor in the auxiliary tank were added into a diluted mixing tank, and the water for injection was added to a defined volume to obtain a suspension drug liquor. 2 ml of the suspension drug liquor was filled into a 2-ml ampoule for sealing.

TABLE 2

| | | | Dose per | Pilot formula |
|---|---|---|---|---|
| | | | ampoule | (g)/100,000 |
| Ingredient | | Function | (mg) | ampoules |
| Concentrated | Pramipexole | Active | 1 | 100 |
| mixing | xinafoate | ingredient | | |
| liquor | Polysorbate 80 | Wetting agent | 0.1 | 10 |
| | Water for | Process water | 250 | 25,000 |
| | injection | | | |
| Auxiliary | Polyvinyl | Suspending | 0.3 | 30 |
| liquor | alcohol 6000 | agent | | |
| | Edetate | Stabilizing | 0.2 | 20 |
| | disodium | agent | | |
| | Sodium | Isoosmotic | 17 | 1,700 |
| | chloride | regulator | | |
| | (for injection) | | | |
| | Anhydrous | pH regulator | 0.56 | 56 |
| | citric acid | | | |
| | Sodium citrate | pH regulator | 1 | 100 |
| | Water for | Process water | 1,746.8 | 100,000 |
| | injection | | | |

### EXAMPLE 4 PREPARATION OF PRAMIPEXOLE BENZOATE INJECTION

For the pramipexole benzoate, a jet mill was used with the feeding speed set to be 400 rpm and the feeding pressure to be 2.1-2.3 bar, and the crushing pressure was adjusted. The pramipexole benzoate was crushed to D₉₀: 3.1 μm; D₅₀: 0.8 μm; and D₁₀: 0.4 μm to different particle size distribution.

Referring to Table 3, the pramipexole benzoate (equivalent to the pramipexole xinafoate) was dissolved in an appropriate amount of water for injection, added into the concentrated mixing tank and sterilized at 121° C. for 15 min. Other auxiliary materials were added into an auxiliary tank, an appropriate amount of the water for injection was added for dissolving, prior to sterilizing by filtration. The drug liquor in the concentrated mixing tank and the auxiliary liquor in the auxiliary tank were added into a diluted mixing tank, and the water for injection was added to a defined volume to obtain a suspension drug liquor. 2 ml of the suspension drug liquor was filled into a 2-ml ampoule for sealing.

TABLE 3

| | | | Dose per | Pilot formula |
|---|---|---|---|---|
| | | | ampoule | (g)/100,000 |
| Ingredient | | Function | (mg) | ampoules |
| Concentrated | Pramipexole | Active | 0.567 | 100 |
| mixing | benzoate | ingredient | | |
| liquor | Polysorbate 80 | Wetting agent | 0.1 | 10 |
| | Water for | Process water | 250 | 25,000 |
| | injection | | | |
| Auxiliary | Polyvinyl | Suspending | 0.3 | 30 |
| liquor | alcohol 6000 | agent | | |
| | Edetate | Stabilizing | 0.2 | 20 |
| | disodium | agent | | |
| | Sodium | Isoosmotic | 17 | 1,700 |
| | chloride | regulator | | |
| | (for injection) | | | |
| | Anhydrous | pH regulator | 0.56 | 56 |
| | citric acid | | | |
| | Sodium citrate | pH regulator | 1 | 100 |
| | Water for | Process water | 1,746.8 | 100,000 |
| | injection | | | |

### EXAMPLE 5 PREPARATION OF PRAMIPEXOLE PAMOATE SUSPENSION INJECTION

For the pramipexole pamoate, a jet mill was used with the feeding speed set to be 400 rpm and the feeding pressure to be 2.1-2.3 bar, and the crushing pressure was adjusted. The pramipexole pamoate was crushed to D₉₀: 2.4 μm; D₅₀: 0.7 μm; and D₁₀: 0.3 μm to different particle size distribution.

Referring to Table 4, the micronized pramipexole pamoate (equivalent to the pramipexole xinafoate) and a wetting agent were added into an appropriate amount of water for injection to be fully wetted, prior to addition into a concentrated mixing tank and sterilizing at 121° C. for 15 min. Other auxiliary materials were added into an auxiliary tank, an appropriate amount of the water for injection was added for dissolving, prior to sterilizing by filtration. The drug liquor in the concentrated mixing tank and the auxiliary liquor in the auxiliary tank were added into a diluted mixing tank, and the water for injection was added to a defined volume to obtain a suspension drug liquor. 2 ml of the suspension drug liquor was filled into a 2-ml ampoule for sealing.

TABLE 4

| | | | Dose | Pilot formula |
|---|---|---|---|---|
| | | | (mg) per | (g)/100,000 |
| Ingredient | | Function | ampoule | ampoules |
| Concentrated | Pramipexole | Active | 1.02 | 100 |
| mixing | pamoate | ingredient | | |
| liquor | Polysorbate 80 | Wetting agent | 0.1 | 10 |
| | Water for | Process water | 250 | 25,000 |
| | injection | | | |
| Auxiliary | Polyvinyl | Suspending | 0.3 | 30 |
| liquor | alcohol 6000 | agent | | |
| | Edetate | Stabilizing | 0.2 | 20 |
| | disodium | agent | | |
| | Sodium | Isoosmotic | 17 | 1,700 |
| | chloride | regulator | | |
| | (for injection) | | | |
| | Anhydrous | pH regulator | 0.56 | 56 |
| | citric acid | | | |
| | Sodium citrate | pH regulator | 1 | 100 |
| | Water for | Process water | 1,746.8 | 100,000 |
| | injection | | | |

EXAMPLE 6 STUDY OF PHARMACOKINETICS OF DIFFERENT PRAMIPEXOLE SALT SUSPENSION INJECTIONS

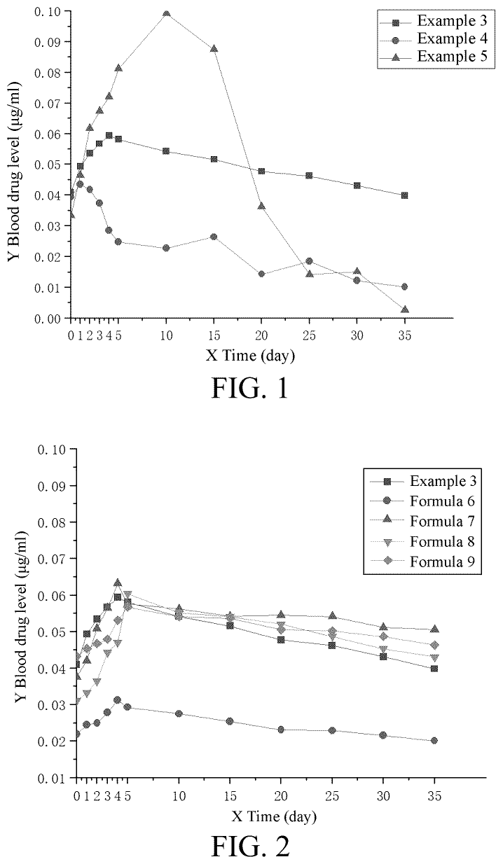

18 adult rats, including 12 males and 6 females (non-perilactation period), were selected and divided into three groups with 6 rats each group (a ratio of males to females of 2:1), and subcutaneously administered with 2 ml of the suspension injections of Examples 3-5 respectively. The main pharmacokinetic parameters of each of test animals were determined: the time to peak, the maximal blood drug level and AUC₀₋ₜ. The determination results are shown in Table 5. Blood drug level curves of each formula are shown in FIG. 1.

TABLE 5

| | Time to peak | Maximal blood drug | AUClast |
|---|---|---|---|
| Formula | (Tmax) (day) | level (Cmax) (μg · ml⁻¹) | (μg · day · mL⁻¹) |
| Example 3 | 4 | 0.0594 | 0.6131 |
| Example 4 | 1 | 0.0435 | 0.6723 |
| Example 5 | 10 | 0.0992 | 0.6326 |

The results shows that the pramipexole xinafoate having a good tendency to release may have a stable blood drug level maintained for a prolonged period of time, with a sustained release for up to 35 days. There are such problems as immediate release and low release concentrations for other salt forms.

### EXAMPLE 7 STUDY OF EFFECTS OF DIFFERENT FORMULAS ON BIOAVAILABILITY OF PRAMIPEXOLE XINAFOATE SUSPENSION INJECTIONS

Based on the formula of Example 3, the types and amounts were adjusted for the pramipexole xinafoate, the wetting agent and the suspending agent to prepare different samples. The formulas are shown in Table 6.

TABLE 6

| Example | | Function | Formula 6 | Formula 7 | Formula 8 | Formula 9 |
|---|---|---|---|---|---|---|
| Concentrated | Pramipexole | Active | 0.5 | 1 | 1 | 1 |
| mixing liquor | xinafoate (g) | ingredient | | | | |
| | Polysorbate 80 | Wetting | 0.1 | — | 0.1 | 0.1 |
| | (g) | agent | | | | |
| | Poloxamer 188 | Wetting | — | 0.1 | — | — |
| | (g) | agent | | | | |
| Auxiliary | Polyvinyl alcohol | Suspending | 0.3 | 0.3 | — | — |
| liquor | 6000 (g) | agent | | | | |
| | PVP (g) | Suspending | — | — | 0.2 | — |
| | | agent | | | | |
| | Sodium | Suspending | — | — | — | 0.1 |
| | carboxymethyl | agent | | | | |
| | cellulose (g) | | | | | |

### Rat Pharmacokinetic Test

24 adult rats, including 16 males and 8 females (non-perilactation period), were selected and divided into 4 groups with 6 rats each group (a ratio of males to females of 2:1), and respectively given with the different formulas at the dose of 20 mg/kg by nasal spray and oral administration (gavage). The main pharmacokinetic parameters of each test animal were determined: the time to peak, the maximal blood drug level and AUC₀₋ₜ. The determination results are shown in Table 7. Blood drug level curves of each formula are shown in FIG. 2.

TABLE 7

| | Time to peak | Maximal blood drug | AUClast |
|---|---|---|---|
| Formulation | (Tmax) (day) | level (Cmax) (μg · ml⁻¹) | (μg · day · mL⁻¹) |
| Example 3 | 4 | 0.0594 | 0.621 |
| Formula 6 | 4 | 0.0312 | 0.3341 |
| Formula 7 | 4 | 0.0632 | 0.6765 |
| Formula 8 | 5 | 0.0604 | 0.6432 |
| Formula 9 | 5 | 0.0568 | 0.6023 |

The results show that the use of different wetting agents and suspending agents has less effect on the release of the product. The absorption amount decreases as the amount of the main ingredient is reduced.

1. A pramipexole xinafoate, having the following structural formula:

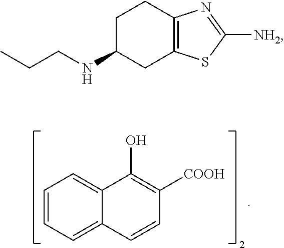

2. A method for preparing the pramipexole xinafoate according to claim 1, wherein a pramipexole is dissolved in a reaction solvent, xinafoic acid is added at room temperature to 60° C. for reaction, and after the reaction is finished, the reaction product is cooled to room temperature and filtered and separated to obtain the pramipexole xinafoate.

3. The method according to claim 2, wherein the reaction solvent is selected from one or more of alcohols, ketones, nitriles, ethers, dichloromethane and dimethyl sulfoxide.

4. A sustained-release pharmaceutical formulation of pramipexole, having the pramipexole xinafoate according to claim 1 as a main pharmaceutical active ingredient.

5. The sustained-release pharmaceutical formulation according to claim 4, wherein the sustained-release pharmaceutical formulation is a capsule, a tablet, an oral suspension, and a suspension injection.

6. The sustained-release pharmaceutical formulation according to claim 5, being in the form of a suspension injection.

7. The sustained-release pharmaceutical formulation according to claim 6, wherein the suspension injection comprises one or more of a wetting agent, a suspending agent, a stabilizing agent, a pH regulator, an osmotic pressure regulator and an analgesic.

8. The sustained-release pharmaceutical formulation according to claim 7, wherein the analgesic is selected from one or more of benzyl alcohol, chlorobutanol, procaine hydrochloride and lidocaine.

9. The sustained-release pharmaceutical formulation according to claim 8, wherein the wetting agent is selected from one or more of polysorbate, polyoxyethylene castor oil, poloxamer 188 and lecithin; the suspending agent is selected from one or more of PVP, gelatin, methylcellulose, sodium carboxymethylcellulose and polyethylene glycol; the stabilizing agent is selected from one or more of sodium sulfite, sodium bisulfite, sodium metabisulfite, sodium thiosulfate and an EDTA disodium salt; the pH regulator is selected from one or more of hydrochloric acid, sodium hydroxide, sodium bicarbonate, an acetic acid-sodium acetate buffer and a citric acid-sodium citrate buffer; and the osmotic pressure regulator is selected from sodium chloride and/or glucose.

10. A method for treating Parkinson's disease, comprising administering an effective amount of the sustained-release pharmaceutical formulation of claim 4 to a subject in need thereof.
